(19) United States
(12) Patent Application Publication
(10) Pub. No.: US 2013/0006154 A1
(43) Pub. Date: Jan. 3, 2013

(54) Control Unit for a Therapy System and Method

(75) Inventor: Mark H. Lowe, Danville, CA (US)

(73) Assignee: CoolSystems, Inc, Concord, CA (US)

(21) Appl. No.: 13/441,767

(22) Filed: Apr. 6, 2012

Related U.S. Application Data
(60) Provisional application No. 61/472,596, filed on Apr. 6, 2011.
(60) Provisional application No. 61/472,598, filed on Apr. 6, 2011.
(60) Provisional application No. 61/472,602, filed on Apr. 6, 2011.

(51) Int. Cl. A61F 7/08; A61H 7/00

(52) U.S. Cl. 601/15; 607/104

(57) ABSTRACT

A therapy system including a control unit for treatment of an animate body. The system includes a therapy wrap including at least one heat transfer device adapted to exchange heat with the animate body and a control unit. The control unit includes an input device for receiving an input signal, a processor for determining a treatment protocol based on the received input signal, and a controller for administering treatment to the animate body using the therapy wrap based on the determined treatment protocol. Also disclosed are methods of administering a temperature-controlled treatment to an animate body.

### CROSS REFERENCE TO RELATED APPLICATIONS

This application claims priority to U.S. Provisional Application No. 61/472,596, filed on Apr. 6, 2011; U.S. Provisional Application No. 61/472,598, filed on Apr. 6, 2011; and U.S. Provisional Application No. 61/472,602, filed on Apr. 6, 2011, which are herein incorporated by reference in their entireties.

### FIELD OF THE INVENTION

The present invention relates generally to therapy of an animate body, and more particularly a system and control unit for providing thermal therapy to a mammal.

### INCORPORATION BY REFERENCE

All publications and patent applications mentioned in this specification are herein incorporated by reference for all purposes to the same extent as if each individual publication or patent application was specifically and individually indicated to be incorporated by reference.

### BACKGROUND OF THE INVENTION

Temperature-controlled therapy has long been practiced in the field for physical therapy, sports injuries, and other settings. Thermal therapy commonly includes cooling and/or heating and applying compression to a traumatized area of a human body to facilitate healing and prevent unwanted consequences of the trauma. This form of therapy is commonly referred to as RICE (Rest, Ice, Compression and Elevation). RICE is also commonly used in sports medicine to reduce the risk of long-term damage to muscles and joints and/or alleviate pain and soreness.

Conventional temperature-controlled therapy involves applying ice bags or the like to a treatment area to provide deep cooling. Elastic wraps are often applied over the bags to keep them in place and provide compression to the body part. Ice bags and elastic wraps lack control and usually require a user to put the bag on and off to adjust cooling.

More sophisticated animate body heat exchangers have been developed recently. Thermal therapy systems commonly include a heat exchanger, a control unit for the heat exchanger, and a sleeve for positioning the heat exchanger on a body part to be treated. The control unit regulates delivery of a heat exchange fluid to the heat exchanger for circulation through a fluid bladder. Many systems also include a compressive mechanism such as a compliant gas pressure bladder that overlays the fluid bladder. The gas pressure bladder directs a compressive force to the fluid bladder to press the bladder against the body part to be subjected to heat exchange and apply compression to the body part to reduce edema.

Existing thermal therapy devices typically use a single control with a wide variety of therapy wraps and heat exchangers. A problem with existing thermal therapy systems is that the user must adjust the therapy manually using the control unit. This allows for user errors and patient compliance issues. Existing systems also often undesirably requires the user to make adjustments based on “feel” and guesswork.

There is a need for improved systems and methods for heating, cooling, and/or compressing a body in need of treatment. There is a need for improved systems for selecting and administering a treatment.

There is a need for a temperature-controlled therapy system with improved patient comfort and/or reduced risks of injury to the body part treated. There is the need for an easy-to-use temperature-controlled therapy system applicable in a variety of settings and environments. There is a need for improved devices for controlling administration of thermal therapy and/or other therapies to a body.

These and other problems are overcome by the invention disclosed herein.

### SUMMARY OF THE INVENTION

The present invention involves improvements in heat transfer therapy apparatus and avoids disadvantages in the prior art.

Various aspects of the invention are directed to a system for providing thermal treatment to an animate body requiring treatment, the system comprising a therapy wrap including at least one heat transfer device adapted to circulate a heat transfer fluid to exchange heat with the animate body; and a control unit. The control unit includes an input device for receiving an input signal; a processor for determining a treatment protocol based on the received input signal; and a controller for administering treatment to the animate body using the therapy wrap based on the determined treatment protocol.

In various embodiments, the determining includes comparing the received input signal to a desired input signal. The processor selects a treatment protocol if the received input signal is the desired input signal and selects another treatment protocol if the received input signal is not the desired input signal.

In various embodiments, the system includes a heat transfer fluid source for supplying heat transfer fluid to the therapy wrap, wherein the controller regulates the supply of heat transfer fluid to the heat transfer device based on the determined treatment protocol. The heat transfer device may comprise a fluid bladder including an inlet, an outlet, and at least one fluidic channel connecting the inlet to the outlet. The heat transfer device may comprise an expandable gas pressure bladder on a side of the fluid bladder opposite the animate body for exerting a compressive force on the bladder. The therapy wrap may be a sleeve including a pouch for receiving the fluid bladder. The therapy wrap may comprise a plurality of heat transfer devices, each of the plurality of heat transfer devices comprising a fluid bladder for circulating a heat transfer medium, the therapy wrap adapted to position the heat transfer devices adjacent the animate body at different locations. The plurality of heat transfer devices may include a first heat transfer device adapted to cover the torso of the animate body and a second heat transfer device adapted to cover an extremity of the animate body, wherein the first heat transfer device is configured to cool the torso of the animate body and modulate the core temperature of the animate body and the second heat transfer device is configured to warm the extremity. The controller may be configured to disable the system if authorization to use the system is not received by the controller. Authorization to use the system may include payment information or authorization of payment information. The controller may be configured to allow the system to be used for a predetermined time period. The controller may be configured to allow the system or wrap to be used for a predetermined number of cycles. The controller may be configured to permanently disable the system or wrap. The predetermined number of cycles may be specified in a patient specific code that is entered before use.

Various aspects of the invention are directed to a system for providing thermal treatment to an animate body requiring treatment, the system comprising a therapy wrap including at least one heat transfer device adapted to circulate a heat transfer fluid to exchange heat with the animate body, and a control unit fluidly connected to the therapy wrap for controlling at least the circulation of heat transfer fluid, wherein control unit is configured to recognize the therapy wrap when the wrap is connected to the control unit.

Various aspects of the invention are directed to a computer program product for use with a control unit having a computer processor and adapted to provide thermal therapy to an animate body using a therapy wrap, the computer program product comprising a computer readable storage medium and a computer program mechanism embedded therein, the computer program mechanism comprising an analyzer for recognizing a therapy wrap having a unique identifier; and logic for selecting treatment settings based on the recognized unique identifier. The computer program may further include logic for regulating pressure therapy of the animate body using the therapy wrap based on the treatment settings.

In various embodiments, the computer program product includes logic for regulating thermal therapy of an animate body using the therapy wrap based on the treatment settings.

Various aspects of the invention are directed to a computer system for use with a thermal therapy system, the computer system including the computer program product above, a computer server, a network, and a thermal therapy system including a therapy wrap and control unit. The therapy wrap and/or control unit communicate with the computer server through the network. The computer program product may be stored on the computer server, the network, and/or the control unit.

Various aspects of the invention are directed to a computer program product for use with a control unit having a computer processor and adapted to provide thermal therapy to an animate body, the computer program product comprising a computer readable storage medium and a computer program mechanism embedded therein, the computer program mechanism comprising instructions for receiving an input signal; instructions for determining a treatment protocol based on whether the received input signal is a desired input signal; and instructions for treating an animate body using a temperature-controlled system having a therapy wrap based on the determined treatment protocol. In various embodiments, the instructions for determining a treatment protocol comprises comparing the received input signal to the desired input signal, wherein the computer program mechanism selects a treatment protocol if the received input signal is the desired input signal and selects another treatment protocol if the received input signal is not the desired input signal.

Various aspects of the invention are directed to a method for treating an animate body in need of treatment, the method comprising applying a first therapy wrap to a portion of an animate body, the first therapy wrap comprising a heat transfer device adapted to circulate a heat transfer fluid to transfer heat with the animate body; connecting the first therapy wrap to a control unit for regulating circulation of the heat transfer fluid in the heat transfer device; treating the animate body using the first therapy wrap under a first set of treatment settings; applying a second therapy wrap to a portion of an animate body, the second therapy wrap comprising a heat transfer device adapted to circulate a heat transfer fluid to transfer heat with the animate body; and treating the animate body using the second therapy wrap under a second set of treatment settings. In various embodiments, the method comprises after the treating with the first therapy wrap and before applying the second therapy wrap, disconnecting the first therapy wrap from the control unit. The treating under the first set of treatment settings and the second set of treatment settings may be the same or different.

The wrap and method of the present invention have other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

FIG. 1 is a block diagram of a thermal therapy system in accordance with the invention.

FIG. 2A is a back view of the therapy wrap in the system of FIG. 1. FIG. 2B is a cross-sectional view of the therapy wrap.

FIG. 3 is a block diagram of the control unit in the system of FIG. 1.

FIG. 4 is a schematic diagram of a portion of the system of FIG. 1, illustrating the flow of data.

FIG. 5 is a flowchart illustrating a method of using the system of FIG. 1, illustrating selection of a treatment protocol.

FIG. 6 is a flowchart illustrating a method of using the system of FIG. 1, illustrating selection of a treatment protocol.

FIG. 7 is a flowchart illustrating a method of using the system of FIG. 1, illustrating a treatment protocol with multiple treatment cycles.

FIG. 8 is a flowchart illustrating a method of using the system of FIG. 1, depicting an error control operation.

### DETAILED DESCRIPTION OF THE INVENTION

Before the present invention is described, it is to be understood that this invention is not intended to be limited to particular embodiments or examples described. Further, when referring to the drawings, like numerals indicate like elements.

Unless expressly stated otherwise, the terms used herein are to be understood as used by one of ordinary skill in the art. In various respects, use of the singular in connection with the terms herein includes the plural and vice versa.

“Body” is to be understood as used in the medical and biological fields and generally includes any animate body including, but not limited to, mammals. In various respects, “body” refers to human or equine patients. In various respects “body part” and “body” are used interchangeably. In various respects, “body part” refers to a part of a body in direct communication with a therapy system as described herein.

“Core cooling” is to be understood as generally used in the medical and biological fields, and in various respects, refers to application of cooling therapy to decrease the body temperature. In various respects, “core cooling” refers to cooling of the internal body temperature of a human below 95 degrees Fahrenheit, and in various respects below 90 degrees Fahrenheit.

“Body temperature” and “internal body temperature” refer to the internal temperature or core temperature of a respective body as understood in the medical art.

As used herein, the “average temperature” of the wrap refers to the average of the wrap inlet temperature and the wrap outlet temperature.

“Temperature delta” refers to the difference between the wrap outlet temperature and the wrap inlet temperature. One will appreciate that in some cases the temperature delta through the wrap depends on the fluid flow rate, the heat load, and the specific heat of the thermal fluid.

“Maximum temperature” and “minimum temperature” generally refer to the maximum and minimum temperatures in a respective element, and in various respects, within the fluid bladder of the wrap.

“Heat transfer fluid” is to be understood as generally used in the art, and in various respects, refers to the fluid circulated in the heat transfer device for exchanging heat with the subject animate body. “Heat transfer fluid”, “heat transfer medium”, “heat exchange medium” and “heat exchange fluid” are used somewhat interchangeably. In various respects, “heat exchange medium” refers to a medium or cooling source through which the heat transfer fluid is passed to lower its temperature before circulating in the heat transfer device.

Various aspects of the invention are similar to the subject matter described in: U.S. patent application Ser. No. 09/127,256 (filed Jul. 31, 1998) entitled, “Compliant Heat Exchange Panel,” issued on Apr. 3, 2007 as U.S. Pat. No. 7,198,093; U.S. patent application Ser. No. 09/798,261 (filed Mar. 1, 2001) entitled, “Shoulder Conformal Therapy Component of an Animate Body Heat Exchanger,” published on Aug. 30, 2001 as U.S. Publication No. 2001-0018604A1; U.S. patent application Ser. No. 09/901,963 (filed Jul. 10, 2001) entitled, “Compliant Heat Exchange Splint and Control Unit,” published on Nov. 8, 2001 as U.S. Publication No. 2001-0039439A1; U.S. patent application Ser. No. 09/771,123 (filed Jan. 26, 2001) entitled, “Wrist/Hand Conformal Therapy Component of an Animate Body Heat Exchanger,” published on Oct. 25, 2001 as U.S. Publication No. 2001-0034546A1; U.S. patent application Ser. No. 09/771,124 (filed Jan. 26, 2001) entitled, “Foot/Ankle Conformal Therapy Component of an Animate Body Heat Exchanger,” published on Feb. 14, 2002 as U.S. Publication No. 2002-0019657A1; U.S. patent application Ser. No. 09/771,125 (filed Jan. 26, 2001) entitled, “Conformal Therapy Component of an Animate Body Heat Exchanger having Adjustable Length Tongue,” published on Oct. 25, 2001 as U.S. Publication No. 2001-0034545A1; U.S. patent application Ser. No. 10/784,489 (filed Feb. 23, 2004) entitled, “Therapy Component of an Animate Body Heat Exchanger,” published on Aug. 26, 2004 as U.S. Publication No. 2004-0167594A1 which is a continuation of U.S. patent application Ser. No. 09/765,082 (filed Jan. 16, 2001) entitled, “Therapy Component of an Animate Body Heat Exchanger and Method of Manufacturing such a Component,” issued on Feb. 24, 2004 as U.S. Pat. No. 6,695,872 which is a continuation-in-part of U.S. patent application Ser. No. 09/493,746 (filed Jan. 28, 2000) entitled, “Cap And Vest Garment Components Of An Animate Body Heat Exchanger,” issued on Jan. 30, 2001 as U.S. Pat. No. 6,178,562; U.S. patent application Ser. No. 10/122,469 (filed Apr. 12, 2002) entitled, “Make-Break Connector For Heat Exchanger,” issued on Mar. 29, 2005 as U.S. Pat. No. 6,871,878; U.S. patent application Ser. No. 10/637,719 (filed Aug. 8, 2003) entitled, “Apparel Including a Heat Exchanger,” issued on Sep. 19, 2006 as U.S. Pat. No. 7,107,629; U.S. patent application Ser. No. 12/208,240 (filed Sep. 10, 2008) entitled, “Modular Apparatus for Therapy of an Animate Body,” published on Jan. 1, 2009 as U.S. Publication No. 2009-0005841A1 which is a divisional of U.S. patent application Ser. No. 10/848,097 (filed May 17, 2004) entitled, “Modular Apparatus for Therapy of an Animate Body,” issued on Mar. 1, 2011 as U.S. Pat. No. 7,896,910; U.S. patent application Ser. No. 11/707,419 (filed Feb. 13, 2007) entitled, “Flexible Joint Wrap,” issued on Nov. 23, 2010 as U.S. Pat. No. 7,837,638; U.S. patent application Ser. No. 11/854,352 (filed Sep. 12, 2007) entitled, “Make-Break Connector Assembly with Opposing Latches,” issued on Jun. 8, 2010 as U.S. Pat. No. 7,731,244, which is incorporated herein for all purposes by reference.

The above systems generally provide active heating, cooling, and/or compression for humans and other animal bodies. They are used, for example, in physical therapy, pre-game conditioning, minor injury care, post-operative care, and emergency medical care, among other applications. Thermal therapy systems exist in a number of different forms. In general, there is a control unit, a connector hose, wrap comprising a heat transfer device and cover, and a power source (i.e., battery or externally-powered electric source).

The therapy wrap comprising the cover and heat exchanger is applied to the portion of the mammal's body to receive therapy. The control unit modulates a heat transfer medium in the wrap to achieve the desired therapeutic result. One such system is disclosed, for example, in U.S. Pat. No. 6,178,562, the disclosure of which is herein incorporated for all purposes by reference.

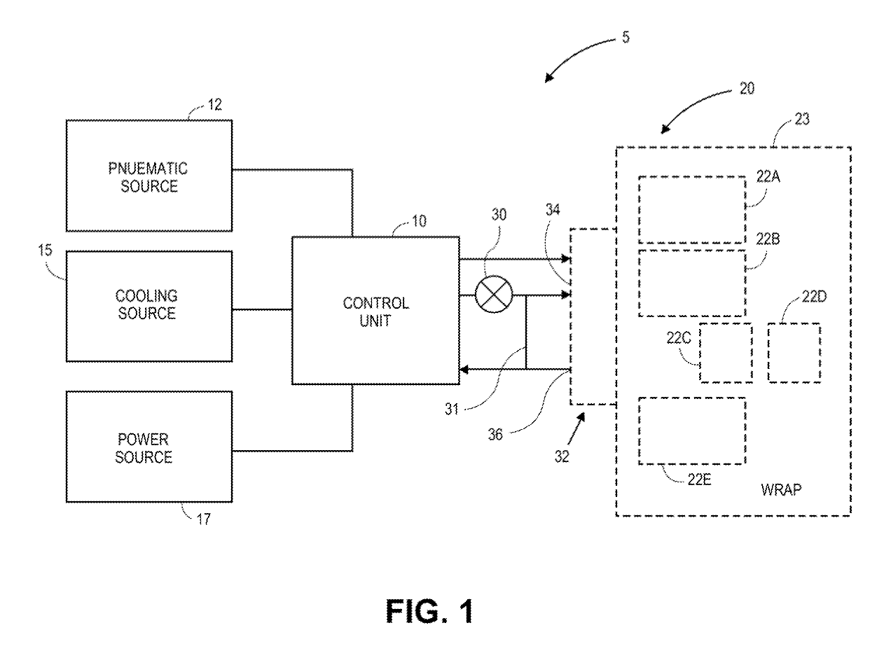

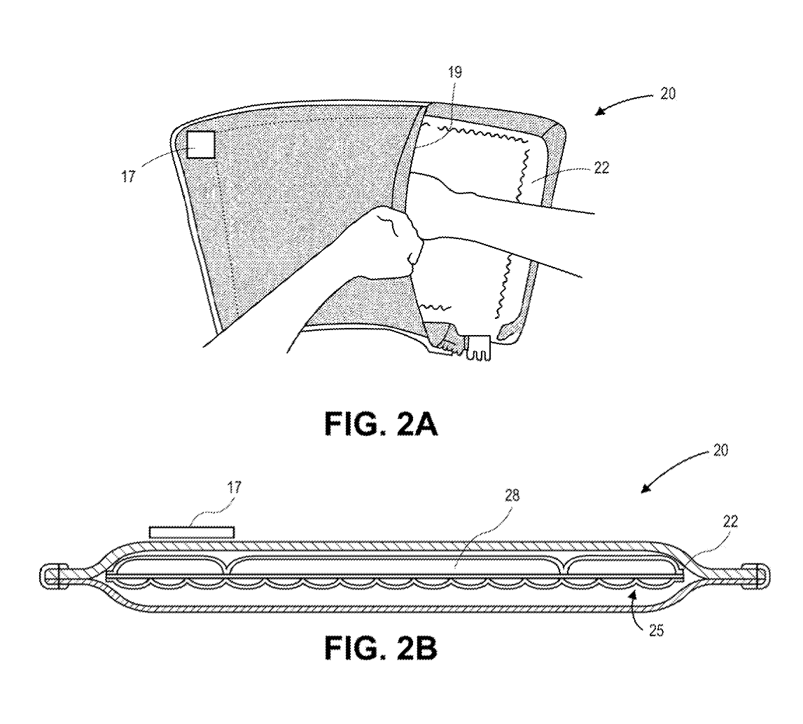

FIG. 1 illustrates a representative number and type of components used in an exemplary thermal therapy system, generally designated 5, in accordance with various aspects of the invention. FIG. 2 illustrates a representative therapy wrap, heat transfer device (heat exchanger), and components for use in the thermal therapy system of FIG. 1. The thermal therapy system is configured for administering temperature-controlled therapy to an animate body through the application of cooling, heating, and/or compression. The therapy wrap of FIG. 2 is similar in many respects to the therapy wraps disclosed in the patents and publications incorporated above.

With reference to FIGS. 1 and 2, thermal therapy system 5 includes a control unit 10, a pneumatic source 12, a heating and/or cooling source 15, and a power source 17. Pneumatic source 12 provides a compressed fluid or gas to a compressive mechanism (e.g. a gas pressure bladder) through the control unit.

Control unit 10 is connected to a therapy wrap 20. The exemplary therapy wrap includes a plurality of heat transfer devices, generally designated 22, and a sleeve 23. Each of heat transfer devices 22 is adapted to transfer heat with an animate body. In various embodiments, the heat transfer devices circulate heat transfer fluid from 15 through a fluid bladder 25 which transfers heat with the animate body.

The therapy wrap 20 of system 5 is configured for wrapping to a portion of an animate body for delivering treatment. The body may include, but is not limited to, a mammalian body such as a human or an equine animal. The exemplary therapy wrap is in the form of a sleeve for connecting various components of heat transfer device 22 to the patient's body. The sleeve is similar in many respects to the sleeve disclosed by U.S. Pat. No. 7,896,910 to Schirrmacher et al. and cover disclosed by U.S. Pat. No. 6,695,872 to Elkins, the entire contents of which patents are incorporated herein for all purposes by reference.

Exemplary therapy wrap 20 includes an opening 19 for directing heat transfer device 22 into a pouch or cavity in the sleeve interior. A portion of sleeve may be pulled back to reveal the pouch and facilitate positioning of the heat transfer device in the pouch as shown in FIG. 2B. Any suitable fastening means can be used to close the opening such as, but not limited to, a zipper.

The pouches may be selectively positioned in predetermined locations on therapy wrap 20. In other words, the pouches may be fixed into a position on the wrap based on parameters defined before use of the wrap. Such parameters may include user preferences or application demands. In various embodiments, the sleeve is configured to position a bladder in one of a plurality of predefined locations. The predefined locations may be determined by user preferences. In various embodiments, the predefined locations correspond to key areas for core cooling of the body.

Therapy wrap 20 may have a variety of shapes and sizes for applying to different portions of the body or different body anatomies. The sleeve may be shaped and configured for application to a mammal, and in various embodiments, a human. In various embodiments, the sleeve is shaped for applying to and covering all or part of a torso, a thoracic region, a cranial region, a throat region, a limb, and a combination of the same. Various aspects of the therapy wrap, in particular the sleeve, shape and design may be similar to the devices disclosed by U.S. Pat. No. 7,107,629 to Miros et al. and U.S. Patent Pub. No. 2005/0256556 A1 to Schirrmacher et al., the entire contents of which are incorporated herein for all purposes by reference.

In general, “heat transfer device” refers to the body heat exchanging component(s). In various embodiments, the heat transfer device includes layers of material defining a flexible fluid bladder through which a liquid is circulated and a gas bladder in which a pressurized gas is injected. Exemplary heat transfer device 22 is in the form of a conventional multi-bladder assembly for positioning adjacent a treatment site of a body. In various aspects, the multi-bladder assembly is manufactured and configured using known techniques. A commonly used thermal bladder assembly uses both a compliant fluid bladder 25 for circulating heat transfer fluid and a gas pressure bladder 28 which overlays the fluid bladder (best seen in FIG. 2B). The gas pressure bladder is adapted to inhibit edema and/or for pressing the fluid bladder against the body part to be subjected to heat exchange.

More specifically, outer gas pressure bladder 28 is adapted to receive a first fluid such as a gas (e.g. air) that can be regulated to provide the desired amount of inflation of the bladder or pressure therein. This inflation or pressure affects the compressive force applied to the animate body during use. Inner fluid bladder 25 is adapted to receive a fluid, such as a coolant which can be in the form of a cold liquid, to transfer heat away from the animate body part. Alternatively, the fluid supplied to the inner bladder can have a temperature higher than the animate body part to heat the body part.

In various embodiments, the heat transfer fluid at the therapy wrap inlet is maintained at a desired temperature. Generally, the desired temperature is lower or higher than the temperature expected for the body part. In a typical cold therapy system, the heat transfer fluid is cooled prior to the inlet to the fluid bladder by passing the fluid through a heat exchanging medium such as cooling source 15. One such system is disclosed, for example, in U.S. Pat. No. 6,178,562, the disclosure of which is herein incorporated for all purposes by reference.

In the exemplary system, cooling source 15 is configured to provide a cooled heat transfer fluid. Suitable cooling sources include, but are not limited to, thermoelectric- and chemical-based cooling. In various embodiments, the cooling source comprises a fluid reservoir cooled with ice, chemical-based cooling devices, or thermoelectric-based cooling devices. For example, a chemical cold pack may be placed into a reservoir of heat exchange fluid. In various embodiments, the cooling source comprises an ice bath and water. In various embodiments, the cooling source comprises a water reservoir cooled by a refrigeration unit. Examples of cooling and heating sources are described in the patents and publications incorporated above and co-pending U.S. App. No. 61/472,596 (attorney matter no. 11185-721.100) filed Apr. 6, 2011, and incorporated herein for all purposes by reference.

Exemplary cooling source 15 is a conventional device for providing cooled heat transfer fluid. The cooling source may include a reservoir physically housed within control unit 10. The reservoir of heat transfer fluid may be cooled by inserting cold elements such as ice or chemical cold packs. In general, a feed liquid is circulated through the reservoir to the heat transfer device inlet. The feed liquid may be circulated through a cooling device such as a refrigeration unit prior to being fed into the reservoir. In various embodiments, the system is a closed loop system and the feed liquid comprises fluid that has been warmed by circulation through the heat transfer device. The fluid may be cooled before circulating back into the wrap. Alternatively, the fluid may bypass the cooling source (e.g. passing through bypass line 31) before recirculating into the heat transfer device. In a practical realization of these embodiments, the heat transfer fluid is normal tap water. In various embodiments, temperature in the reservoir is in a range between 40° F. and 50° F. Other direct and indirect cooling sources may be provided such as thermoelectric, chemical, and electromechanical devices as would be understood by one of skill in the art.

The exemplary return system includes a return line to return the heat transfer fluid in the heat transfer device back to cooling source 15. Additionally or alternatively, the return system may include valves, diverters or other flow control elements. The cooling source may include one or more reservoir inlets or reservoir outlets, a baffle, a filter, a diffuser and the like to adjust performance.

Performance of thermal therapy device 20 may be improved by adjusting the heat transfer fluid flow rate, adjusting the heat transfer device temperature, and/or providing additional features to the thermal therapy device. In a closed loop, return flow arrangement such as that shown in FIG. 1, by example, the velocity of the fluid and the heat transfer rate are generally proportional to the flow rate. Reducing the flow rate of the fluid of a given temperature through heat transfer device 22 will also reduce the amount of energy removed from (or added to) the patient. Conversely, increasing the flow rate will increase the amount of energy removed from (or added to) a patient. In a cold therapy device, with the wrap applied to a mammalian body, the temperature of the fluid leaving the wrap is warmer than the temperature of the fluid entering the wrap because the mammalian body is typically warmer than the thermal fluid. The temperature from cooling source 15 determines the inlet temperature of heat transfer device 22 and is generally lower than the average temperature in the wrap. For example, if an average wrap temperature of 5° C. is desired, then the inlet temperature may be about 4° C. In this example, the outlet temperature may be about 6° and the temperature delta across the heat transfer device may be 2° C.

Further details regarding fluid bladders, cooling sources, and their operation and manufacture are described in U.S. Pat. Nos. 7,198,093 and 6,695,872, both to Elkins, U.S. patent application Ser. No. 12/939,986 to Lowe and co-pending U.S. App. No. 61/472,596 (attorney matter no. 11185-721.100) filed Apr. 6, 2011, the entire contents of which are incorporated herein for all purposes by reference.

Exemplary therapy wrap 20 includes a plurality of heat transfer devices 22a, 22b, 22c, 22d, and 22e. The heat transfer devices are attached to the therapy wrap using conventional techniques. One will appreciate from the description herein that therapy system 5 may be modified to include many combinations of therapy wraps and heat transfer devices. The system may include one therapy wrap with one heat transfer device. The system may include a plurality of therapy wraps. Each of the plurality of therapy wraps may include one heat transfer device or a plurality of heat transfer devices. Thus, the system is easily scalable.

Exemplary system 5 includes therapy wrap 20 having a plurality of heat transfer devices of different sizes and locations. The heat transfer devices are configured to provide different levels of thermal therapy, in other words, different heat transfer rates. The therapy wrap may include a plurality of heat transfer devices, each one administering treatment under different conditions. The different treatments may be applied essentially simultaneously.

Any or all of the heat transfer devices may include a compressive gas pressure bladder. The number, type, and location of the heat transfer devices to be positioned on the body may be based on the desired treatment as would be understood by one of skill in the art. If core cooling of the body is desired, for example, multiple wraps may be provided for each of the key cooling points to lower the body temperature. Each of the multiple wraps may include one or more heat transfer devices. The selection and configuration of the wraps may be modified to enable wrapping to the target parts of the body and positioning of selected heat transfer devices. For example, a heat transfer device with higher heat exchange capabilities may be selected for a chest region than a heat transfer device for an arm.

Therapy wrap(s) 20 and/or heat transfer device(s) 22 may be connected to control unit 10 in various manner. These components may be connected in serial (e.g. a daisy chain), in parallel, or a combination of the same. In exemplary system 5, the heat transfer devices 22a, 22b, 22c, 22d, and 22e are connected in parallel to control unit 10.

In the exemplary embodiment, control unit 10 is connected to therapy wrap 20 through a pump 30. Pump 30 is in communication with cooling source 15 and therapy wrap 20. The pump delivers heat transfer fluid from cooling source 15 to the therapy wrap through connector 32. In general, the pump is controlled by the control unit using a control signal.

Exemplary system 5 includes valving and a bypass line 31 connecting the inlet and outlet fluid lines to therapy wrap 20. Bypass line 31 allows a portion or all of the cooled fluid from cooling source 15 to bypass the wrap. This can be useful for shutting off the flow of fluid to the therapy wrap. This may also be useful for adjusting the flow of fluid through the wrap, for example, by siphoning some of the cooled flow from the wrap inlet and recirculating flow in the wrap to increase the average temperature in the wrap.

Exemplary connector 32 provides one or more connections to the control unit in a single connector. The exemplary connector 32 has fluid ports, a gas port, and an electrical connection. The fluid ports include an inlet 34 and an outlet 36. The connector includes a single gas port for injecting and exhausting a pressurized gas from the gas pressure bladder. The exemplary fluid and gas ports are generally connected by the fluidic connection inlet and outlet. Connector 32 connects to the control unit with hoses and/or other conventional fluidic devices. The connector for the fluid and gas ports is similar in many respects to the three-port manifold connector disclosed by U.S. Pat. No. 6,871,878, the entire contents of which is incorporated herein for all purposes by reference. Other suitable manifold constructions are disclosed in U.S. Pat. Nos. 5,104,158 and 5,052,725, both to Meyer, et al. and both hereby incorporated herein for all purposes by reference. It should be understood that other manifold configurations and/or couplings to provide fluid flow between the fluid source and the bladders can be used as would be apparent to one of skill in the art.

In various embodiments, the connector includes a valve in the fluid inlet and fluid outlet passageways. Suitable valves include a spring-loaded valve to allow the selective passage of fluid therethrough or a check valve. The valves may allow flow when the fluid hose connectors are coupled to the manifold and prevent fluid flow when the fluid hose connectors are uncoupled from the manifold as would be understood by one of skill from the description herein. In this manner, fluid such as a liquid coolant is blocked from exiting the fluid bladder when the fluid hoses are uncoupled from the manifold. In various embodiments, the valves are controlled by an actuator and control unit 10. Thus, the control unit may control the fluid flow by opening and closing the valves based on control parameters.

In addition to controlling the flow of heat exchange medium to the fluid bladder 25, exemplary control unit 10 controls the flow rate and pressure of gas supplied to the compressive gas bladder 28 to control inflation, deflation, and compressive pressure. In various embodiments, the gas delivered to gas pressure bladder 28 is compressed air.

In various embodiments, the pressure of gas furnished by the control unit is between about 0.25 psig and about 20 psig, preferably between about 0.25 psig and about 5 psig, and more preferably about 0.25 to about 1.5 psig. In various embodiments, the control unit maintains a compressive force of between about 0.25 psig and about 5 psig. In various embodiments, the control unit maintains a compressive force of between about 0.25 psig and about 0.5 psig. In various embodiments, the pressure of gas furnished by the control unit is user selectable in increments of 5 mm Hg from 0 mm to about 75 mm. In various embodiments, the system includes a plurality of gas pressure bladders, a first gas pressure bladder at a first pressure and a second gas pressure bladder at a second pressure different than the first.

In various embodiments, the pressure of gas furnished by the control unit is based on the patient's response. For example, if the patient is wearing the wrap during exercise, the pressure may vary based on how strenuous the exercise is. If the patient is having trouble breathing, the control unit may decrease the compressive force around the lungs. The pressure profile map may be set to adjust based on a predetermined routine. In various embodiments, the pressure profile map includes 3 minutes of slowly increasing pressure followed by 2 minutes of decreasing pressure. In various embodiments, the pressure profile map includes 30 seconds of increasing pressure followed by 15 seconds of decreasing pressure. In various embodiments, the pressure fluctuates at random. In various embodiments, the pressure profile map includes 2 minutes of compression followed by 1 minute with no compression.

The strength and frequency of the pulses may be modified depending on the application. In various embodiments, the control unit delivers pulses of compression for massaging therapy.

In various embodiments the wrap can be used with a rigid or semi-rigid support such as a brace. In various embodiments, the control unit can apply and maintain a low pressure or no pressure when the control unit detects a brace in use with the wrap. In various embodiments, the control unit can apply and maintain higher pressures when the control unit detects a brace not in use with the wrap. In some embodiments, a low pressure is less than 10 psig, 5 psig, 4 psig, 3 psig, 2 psig, 1 psig, or 0.5 psig. In some embodiments, a high pressure is greater than 0.5 psig, 1 psig, 2 psig, 3 psig, 4 psig, 5 psig, or 10 psig.

One or more electrical connections are provided on exemplary connector 32. The connections are similar in many respects to conventional electrical panels. The exemplary electrical connections allow an electrical signal to be delivered through a line 45 from control unit 10 to optional electrical components on therapy wrap 20. For example, the control unit may power an on-board display on the wrap. In another example, the control unit powers a solenoid compression mechanism in place of a gas pressure bladder.

In various embodiments, therapy wrap 20 and control unit 10 are configured to perform a “handshake.” Various aspects of the systems for performing the “handshake” are similar to conventional systems and will be appreciated by one of skill from the description herein.

The therapy wrap may be configured to be automatically recognized by the control unit. In various embodiments, one of the control unit and therapy wrap includes a circuit for communicating identifying information related to the wrap to the control unit. Each type of therapy wrap (e.g. a model number, product category, or SKU) may have a unique identifier. Accordingly, the control unit can recognize the difference between two therapy wraps with the same design and configuration. The exemplary wrap includes an identifier unique to the specific wrap to allow the control unit to customize treatment. The exemplary unique identifier is embodied in a tag device 17. For example, the tag device may include a circuit, chip, label, or device comprising the unique identifier.

In various embodiments, connection of the therapy wrap to the control unit establishes a communications link. For example, a communication line may be provided with the electrical connection described above. The system includes a handshake protocol and handshake circuit for processing one or more handshake signals from the therapy wrap. Existing handshake processing circuitry may be used. In the case of multiple therapy wraps and/or multiple heat transfer devices, the system may include a multiplexer for routing the handshake signals.

In various embodiments, therapy wrap 20 includes a unique identifier to be recognized by the control unit. The control unit may include a reader for reading the unique identifier. The unique identifier may include, but is not limited to, a radiofrequency identification (RFID), a bar code, a digital signature, a mechanical or electromechanical signature, a USB dongle, and more. In another example, the unique identifier is an indicator on the product label that is read by the user and entered into the system user interface. One will appreciate that other variations and modifications of the handshake may be employed. For example, the unique identifier may be associated with the heat transfer device instead of the therapy wrap.

In various embodiments, the therapy wrap and control unit include a Hall effect sensor. The therapy wrap includes a magnet in a unique orientation. When the wrap is placed next to the control unit, the control unit can identify the wrap by the resulting voltage or current.

In various embodiments, control unit 10 includes a writer to write to therapy wrap 20. The therapy wrap may include memory or a digital signature. Accordingly, the control unit may write or overwrite the data on the wrap. The control unit may also be configured to create a physical label such as a sticker for placement on the wrap. The label may include selected data to be referenced by a user or clinician later.

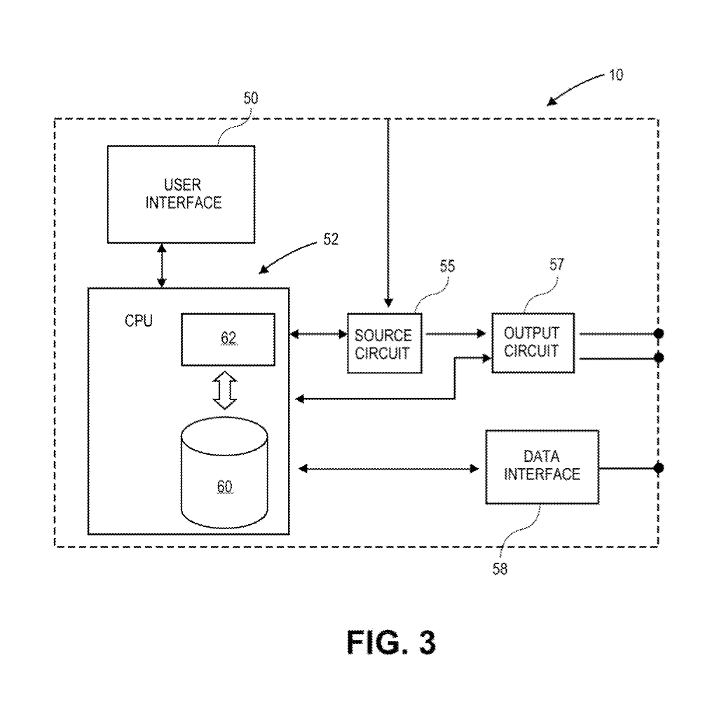

In various embodiments, the control unit user interface 50 (shown in FIG. 3) allows a user to manually enter information identifying the therapy wrap. For example, the control unit may include a keypad for entering the therapy wrap serial number, a patient identification number or code, or a wrap identification number or code.

The “handshake” procedure described above opens a variety of possibilities for operation of system 5. Once the control unit recognizes the therapy wrap, the control unit can customize treatment based on the wrap. The handshake procedure can also allow for tracking and control of individual therapy wraps. This procedure can be used to limit the number of uses of the wrap, for example, when an expected maintenance point has been reached or regulatory or hygiene requires replacement of a therapy wrap. In one example, the control unit tracks the number of wrap uses and shuts down after a preset number. In another example, the control unit checks for authorization to use the wrap, control unit or system and can disable the device if proper authorization is not detected. In some embodiments, authorization can include payment authorization which enables the end user to use the device for a limited number of treatments or time. In some embodiments, a patient specific number or code that specifies the duration or number of uses can be entered before use. In some embodiments, the wrap and/or control unit can include a GPS chip that allows the device to be tracked, which can be useful for retrieving the device from the end user due to nonpayment or any other reason. The control unit can also include programming to confirm the proper therapy wrap is being used based on the treatment settings to avoid user errors. The control unit can perform the handshake data processing locally, or the control unit can communicate with an external network, for example, through an Internet connection. The data can be communicated with the control unit in real-time.

In some embodiments, the therapy wrap can have a microchip that handshakes with the control unit. Once the handshake is established, the control unit marks and/or stores the date and time, which can also be stored on the wrap. In some embodiments, the therapy wrap can be used for a predetermined time period, such as 1, 2, 3, or 4 weeks, after the date and time of the initial handshake. Thereafter, each time the wrap handshakes with any control unit, the control unit can determine the time remaining from the date stored on the wrap, and if the control unit determines that the end of the predetermined time period has passed, the control unit does not allow the wrap to be used.

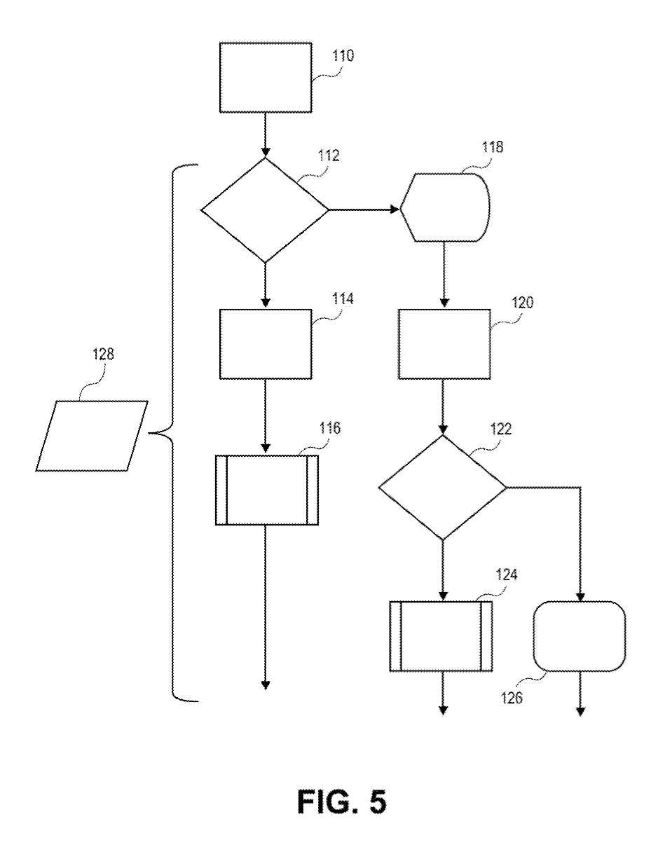

Further details regarding operation of the control unit and therapy wrap will be understood from the description below with reference to FIGS. 5-8.

Exemplary system 5 includes a single control unit 10, pneumatic source 12, cooling source 15, and power source 17. One will appreciate, however, that the number and type of components of the system may vary. The system may include a plurality of pneumatic, coolant, and/or power sources. The system may include a plurality of control units. In various embodiments, the system includes a dedicated pneumatic, coolant, and/or power source for each control unit. In various embodiments, the system includes a dedicated control unit for each therapy wrap.

FIG. 3 illustrates a number of representative components of control unit 10. The exemplary control unit includes a user interface 50, a central processing unit (CPU) 52, and several other input/output components. The user interface may be implemented with conventional input/output devices, including, but not limited to, a display, speaker, input keys, a touchscreen, a button, an alarm, a knob, a dial, and/or a microphone. The input and output components may include other components as would be appreciated by one of skill from the description herein.

Exemplary control unit 10 includes a source circuit 55, a controller or output circuit 57, a user interface 50, and a data interface 58. The exemplary source circuit receives power from power source 17 (shown in FIG. 1). The source circuit may include a transformer and other known devices. In general, the exemplary source circuit and/or output circuit include circuitry for controlling one or more of the flow of heat transfer fluid, gas, and electrical power to heat transfer device 22 (shown in FIG. 1). Exemplary controller or output circuit 57 outputs a control signal to the respective components of the system such as the pump to regulate the flow of heat transfer fluid to the heat transfer device. The output circuit also generates a control signal to activate bypass line 31 as described above (shown in FIG. 1). Depending on the application, one will appreciate that other components may be included in the control unit such as a pulse width modulation (PWM) controller, an analog-digital converter (ADC), an isolation transformer, a filter, and more.

Exemplary control unit includes a CPU 52, memory 60, and software 62. The CPU may be a microprocessor such as a model 68332 available from Motorola. One will appreciate that other processor units may be utilized depending on the application requirements.

Exemplary memory 60 includes random-access memory such as a DRAM (dynamic random access memory) or SRAM (static random access memory), and nonvolatile memory such as an EEPROM (electrically erasable programmable read-only memory). The EEPROM can be used to store software programs executed by the microprocessor to control operation of control unit 10, as would be understood from the description herein. The EEPROM may allow the stored software programs to be remotely updated, for example, by downloading updates through a communications port or the Internet of data interface 58.

Software 62 may include, but is not limited to, proprietary software and modified versions of existing medical device software. The software may be implemented in various forms such as embedded on the processor or in the memory. The software may also be loaded onto a computer-readable medium such as a CD or flash drive or provided. In various embodiments, the software comprises firmware.

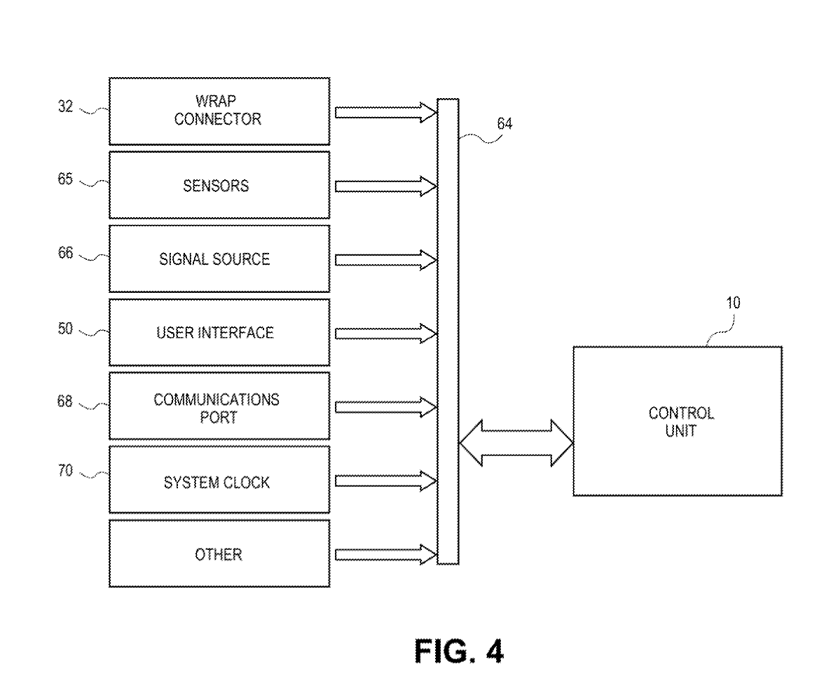

FIG. 4 is a schematic diagram illustrating the flow of data in exemplary system 5, and in particular, between control unit 10, a data network 64, and the other respective components. The exemplary control unit acquires or receives data from a number of information sources including, but not limited to, connector 32, sensors 65, a signal source 66, user interface 50, a communications port 68 part of data interface 58, system clock 70, and/or others sources. The sources of input information to the control unit may relate to the system and/or patient. The control unit may also output data to any of the sources through data network 64.

In various embodiments, control unit 10 includes an internal data acquisition unit for acquiring data relating to the system and/or patient from sensors. In one embodiment, exemplary source circuit 55 includes transmit and receive circuitry (not shown) such a signal generator, switch, and a signal generator control.

In various embodiments, control unit 10 receives an input signal from an information source and outputs a control signal. The control system processes the input signal based on an algorithm or logic to determine a result. In various embodiments, the control system adjusts operation of the therapy wrap based on the result.

As described above, exemplary connector 32 (shown in FIG. 1) may provide information about the specific therapy wrap connected to the control unit. Information related to operation of the therapy wrap may be routed through the connector.

In various embodiments, system 5 includes sensors 65 and/or a signal source 66. Suitable sensors include, but are not limited to, a temperature sensor, a pressure sensor, and a flow rate sensor. The sensors may also include sensors for monitoring the patient including, but not limited to, an EKG sensor, a pulse oximeter, and a blood pressure monitor. The patient-related sensors may be applied to the patient with therapy wrap 20, for example, by holding against the skin of the body with the wrap.

In various embodiments, the system includes sensors to monitor the system performance. Sensors 65 acquire data and communicate with control unit 10 using otherwise conventional techniques. In an exemplary embodiment, temperature sensors are positioned in the fluidics of the thermal therapy device and/or the cooling source. A pressure sensor may be used to monitor pressure in the compressive device. In another example, the control unit may receive a temperature reading at an outlet of the heat transfer device and determine that inlet temperature needs to be decreased.

Information from signal source 66 may be derived from a number of sources. The signal source information may include, but is not limited to, feedback and monitoring information from the wrap and the control system. Signal sources may include monitors connected to the system components such as pump 30. Signal sources may include data output by separate controllers associated with various components. The control unit may extract information from components that include basic circuitry. For example, the pump, valving system, power source, pneumatic source, and cooling source may include basic circuitry that controls operation of the respective device. The control unit can include a data extractor for extracting data related to operation of the devices. The data extractor may be provided on the device being monitored. The acquired information may be stored on the respective component. The information may be acquired by the control unit at specified intervals, or the information may be continually communicated to the control unit such as through a communications link.

User interface 50 allows for receipt of manual information from a user as described above. The user input may include, but is not limited to, information related the desired therapy, the patient, and the system components being used. The desired therapy information may include, but is not limited to, the type of therapy (e.g. hot, cold, and/or compression), desired treatment time, desired average temperature in the therapy wrap, adjustments to the temperature, desired compression, adjustments to the compression, and patient characteristics such as fitness level, height, weight, and sensitivity. The user may include those other than the person or animal receiving the treatment. For example, a caregiver may select a customized therapy setting or input a patient indication such as ligament tear or cardiac arrest.

Communications port 68 may receive information from various sources. In an exemplary embodiment, the control unit includes a flash memory reader to allow a user to download information to the control unit. The communications port may allow the control unit to communicate with a central server or other control unit. The control unit software may also be updated via the communications port. The communications port may include, but is not limited to, a wireless connection, an Internet connection, infrared, a disk drive, and/or other components understood by one of skill.

In various embodiments, a physician can prepare a treatment protocol on an external computer and the protocol may be downloaded to the control unit using, for example, data communications port 68 or a recordable medium (e.g. a flash drive or compact disc). The data communications port may also be used for other activities such as payment processing. For example, the end user can pay or prepay per use or number of uses or time period of use. When the communication port 68 is used to connect the device to the internet, the device can be updated that payment has been received, allowing the device to be used for the appropriate number of uses or cycles or time period. The device can be updated automatically regarding the payment by an online payment processing server, for example, or the end user can be provided with an activation code that can be entered into the device. Alternatively, payment information can be transferred to a dongle which can be used to transfer the payment information to the device, or payment information may be transmitted wirelessly to the device via an RFID tag or other encoded transmission. In some embodiments, the payment information is received by the controller and serves as authorization for the end user to use the device or system as prescribed herein.

In various embodiments, control unit 10 logs the time associated with acquired information. For example, it may desirable to log the time a sensor reading was taken. In various embodiments, the control unit stores information related to unusual patient responses and the time of the events. The control system may, for example, note the time when a blood pulse quickens above a predetermined level or changes drastically. The control system may log the amount of time it takes to decrease the core body temperature to a desired temperature. In various embodiments, the control unit determines the time values using system clock 70.

The data acquisition may be performed at intervals or on an essentially continuous basis. In various embodiments, the data acquisition function is performed predetermined points in time. In various embodiments, the data acquisition function is performed in real time. The control unit may also acquire data based on specific events. For example, the control unit may be programmed to acquire temperature readings when the flow of heat transfer fluid begins and at set intervals thereafter. The control unit may be programmed to acquire certain data based on patient response. For example, the control unit may acquire specified system operational information and patient vital signs if the patient's heart rate or body temperature unexpectedly drops.

In various embodiments, the data acquired by the control unit is displayed to a user on an external output device. The output device may be a digital display, illuminated lights, or the like.

In various embodiments, the system logs information from one or more of the input sources and stores the information in memory 60. The logged information may be used for feedback control, error logging, diagnostics, performance optimization, and the like. For example, the sensors and processing may be configured to recognize blockage in the heat exchanger if the flow rate drops below an expected minimum threshold level. The information may also be used for troubleshooting. In various embodiments, control unit 10 processes the input information to operate components of system 5 to produce a desired therapeutic result. In various embodiments, the data is stored for later analysis by a clinician, customer support specialist, technical support, and the like.

The logged information may include an indication that the component has been used and under what conditions. This information can be stored in memory for later retrieval, for example, to pass to a doctor once a patient reaches the hospital. This information can also be used to restrict use of the components. For example, the system may deliver a signal to activate a shut-off valve or other mechanism to prevent re-use of disposable components or other undesirable re-use of components. In various embodiments, the system logs the number of times the respective components have been used so time to replacement can be estimated and tracked.

In an exemplary embodiment the control unit is configured to perform a diagnostic routine. A stop valve or similar device is installed inside the control unit to turn the flow of heat transfer fluid on and off. The control unit tests the flow rate and/or fluid pressure without the wrap connected. Next, the control unit closes the shunt valve and measures the flow rate and/or fluid pressure. The control unit then compares the data to identify and diagnose problems. In various embodiments, a shunt valve is installed in the control unit to lock down the control unit. By lock down, it is meant that the control unit cannot be unlocked and used without a key, such as a password.

In various embodiments, the system acquires data from sensors and the information is processed for diagnostic or troubleshooting functions. For example, the system may determine that there is a leak or a blockage (e.g. a kink or obstruction in the fluid pathway) if the fluid flow rate drops below a predetermined threshold. The system may determine that there is a blockage if the backpressure rises above a predetermined threshold.

The acquired information input to control unit 10 can also be used to derive information about the performance of system 5 and the patient's response. The control unit may process the input information to determine or perform one or more of the following: verify performance, anticipate user issues, design treatment protocols, aid troubleshooting, calculate wattage removed (heat transfer) during treatments, verify patient compliance, and calculate hours of operation. The resulting determined information may be fed back into the control unit to optimize performance. Information that can be input to the control unit includes, but is not limited to, heat transfer device inlet temperature, heat transfer device outlet temperature, cooling source inlet temperature, cooling source outlet temperature, heat transfer fluid flow rate, elapsed time, system errors, and combinations of the same. The system may also log flow rate and pressure anywhere in the heat transfer fluid system. Sensors 65 may also be selectively positioned on the body to measure or estimate core temperature.

In various embodiments, system 5 may be controlled manually by a user or automatically. The system may be configured to operate based on and off modes. Alternatively, the system may utilize control unit 10 to monitor and/or regulate fluid flow through pump 30 and therapy wrap 20.

With reference to FIGS. 1 and 5, an exemplary embodiment of a method of using the system in accordance with the invention will now be described. The method will be described in connection with a system for administering cooling therapy and compression; however, the following description is not intended to be exhaustive or to limit the invention to the precise forms disclosed. To the contrary, one will appreciate that many modifications and variations are possible.

Although the method of the invention will be described in terms of cooling therapy, one will appreciate that the methods and systems of the invention may be configured for administering a variety of treatments to a patient in a variety of settings. Suitable treatment settings include, but are not limited to, a clinic such as a rehabilitation or physical therapy clinic, an operating room (OR), a post-operative setting, a hospital, emergency medical care, and more. Suitable treatments include cooling therapy, heat therapy, compression therapy, and combinations of the same. The control unit may also be used to control administration of other treatments and functions commonly used in the above treatment settings.

FIG. 5 is a flowchart illustrating administration of a customized treatment profile for a patient. The system may be preprogrammed to administer a variety of therapy procedures. One of skill will appreciate that, if the system recognizes what type of components are being used, the system can automatically tailor operation to the component. This information can be used in combination with the patient information. In general, a user may select a specific therapy wrap, heat transfer device, and working element from among a set of components. The system then recognizes which components have been selected and provides the associated, customized treatment. For example, a specific heat transfer device and/or therapy wrap for treating a patient undergoing surgery can be selected by a physician. When the components are plugged into the system, the system can recognize that the patient requires thermal treatment for a particular surgery and proceed to deliver treatment best tailored for that patient. In various embodiments, the system includes a plurality of treatment routines stored in a database and selects one of the routines. The selection may be done automatically or with user input. The selection may change based on the patient's response to treatment.

Turning to FIGS. 1 and 5, at block 110, therapy wrap 20 and the patient are prepared for the administration of treatment prior to operation of system 5. The therapy wrap is connected to the system by attaching connector 32 to control unit 10.

Block 110 includes applying therapy wrap 20 to a treatment area of the patient's body. The exemplary therapy wrap comes pre-assembled with a fluid bladder and gas pressure bladder. As noted above, the therapy wrap may take different forms such as a vest, a wrist wrap, a head cap, a wrap for a body limb, and more. Exemplary therapy wrap 20 is designed and configured to be applied to the chest region of the patient. The wrap may be applied to the body before or after connecting the wrap to the control unit.

At block 112, control unit 10 performs a “handshake” operation with therapy wrap 20. The control unit programming interrogates the therapy wrap for a unique identifier value at block 110. Next, at block 112 the control unit checks whether it recognizes the returned value.

In various embodiments, the control unit includes a library or table of recognized identifiers. If the returned unique identifier value is found in the table of identifiers, the handshake is complete, the therapy wrap has been recognized, and the system proceeds to block 114. If not, the system proceeds to block 118.

In the exemplary method, if the control unit recognizes the unique identifier the control unit will acquire information related to the individual wrap and the wrap characteristics. For example, the control unit may recognize that the connected wrap belongs to user “John Smith” at “Address 1”. The control unit may recognize that the wrap has been used X out of an allowed N times where X is more than zero and less than N. The control unit may also track the number of uses in fractions, for example, where only a part of a treatment is completed before the unit turns off. In the exemplary method, the control unit recognizes the type of wrap (e.g. a chest wrap) and the components in the wrap (e.g. a fluid bladder and gas pressure bladder of known shapes and dimensions). Some or all of this information may be stored in the control unit. For example, the control unit may include a data file with product specifications for every type of wrap such that the control unit can call on a specific wrap's specifications once it is recognized.

At block 114, the control unit selects a treatment based on the recognized unique identifier value. In various embodiments, the control unit includes a look-up-table (LUT) having a list of treatment protocols. Each unique identifier may have a corresponding one or set of treatment protocols associated with it. In various embodiments, the unique identifier is one of several values in an input set used to select a treatment protocol. The input set may include, for example, a unique identifier value, one or more user input values, one or more patient characteristics values, and/or other input values discussed herein. The control unit may select a treatment protocol associated with the input set using an LUT. Other techniques understood by those skilled in the art may be employed to select a treatment from among a plurality of treatment options based on an received value may be used.

In various embodiments, a plurality of treatment protocols are predefined and stored in the control unit memory. In an exemplary embodiment, the control unit prompts the user for information and the control unit designs a treatment by processing the user answers (user input) using the software. The software may include an algorithm for selecting one of a number of predefined treatment protocols based on the combination of inputs.

In various embodiments, the system includes a treatment wizard. The system may be pre-loaded with a treatment algorithm and selects a treatment routine based on a user's response to various questions. In various embodiments, the system includes programming for customized thermal therapy treatment. In one example, the user inputs the following information: therapy wrap location=“knee”, activity=“sports,” and treatment=“physical therapy” (PT). The control unit then determines based on the system clock that it is a weekend during the day and thus filters the treatment protocols for those associated with a rest period and the user input. The control unit then processes the inputs with an algorithm to select a treatment protocol. The control unit may filter the entire set of predefined protocols by the user inputs, rest period, and known wrap configuration before processing with the algorithm. In this example, the control unit selects a treatment protocol of full cold, 30 minutes on, 30 minutes off associated with inputs “knee”, “physical therapy,” rest period, etc. To the contrary, if the user entered “sprain” instead, the treatment protocol might be medium temperature, 20 minutes on, 20 minutes off. In various embodiments, the treatment comprises strong pulses of compression for massaging therapy.

Software 62 algorithm may be similar to readily available software algorithms. The exemplary algorithm processes the input values based on known associations with treatment parameters. For example, the algorithm may incorporate the fact that compression is best in the range of about 0.25 psig to 0.5 psig, preferably below 0.5 psig when treating the chest region so as not to inhibit breathing. For an unconscious patient, the algorithm may incorporate the fact that no compression is desired. Similarly, the algorithm may incorporate known associations between flow rate, therapy wrap inlet temperature, compression, and more with the treatment location with respect to the body, desired treatment or patient ailment, patient condition, and patient physique. In various embodiments, the algorithm assigns weights to these factors and associations. In various embodiments, the algorithm comprises a summing of the above functions (associations) to select a “best” treatment.

In various embodiments, control unit 10 adjusts the flow rate to the heat exchanger (e.g. fluid bladder) and/or the inlet temperature based on an anatomical feature and/or patient response. The control unit may be configured to increase the flow rate, thus increasing cooling, in areas requiring more cooling and decrease the flow rate in areas requiring less cooling. The control unit may increase the flow rate in a region of the wrap adjacent soft tissue. The control unit may decrease the flow rate in a region adjacent bone. The control unit may control the flow rate and temperature based on the known properties of the wrap and its intended use. For example, the control unit may take into consideration the vasculature and other physiological characteristics of the patient. In the case of core body cooling, the control unit may increase cooling around selected blood vessels. As discussed above, the control rate and temperature of the fluid affect the rate of heat exchange. Thus, an equal amount of heat exchange can be achieved by increasing the flow rate and lowering the temperature delta in all or a portion of the heat exchanger. In various embodiments, the control unit increases the flow rate and lowers the temperature in certain regions of the therapy wrap. For example, in a region where the patient's body is more temperature sensitive, the control unit can increase the minimum temperature while achieving the same amount of heat exchange by increasing the flow rate.

One will appreciate from the foregoing other modifications and variation of the system. For example, the software, treatment protocol data, and unique identifier data, among other things, may be located on an external device instead of the control unit. The processes may be performed, for example, on an external computer and the results may be communicated to the control unit.

In an exemplary embodiment, control unit 10 is configured to administer thermal therapy using a therapy wrap for a patient undergoing surgery. The exemplary method includes prompting the user for information, for example, body part, kind of surgery, and time since last surgery and/or injury. The exemplary method includes prompting the user for physiological parameters of the patient such as body mass index (BMI), height, weight, gender, and/or fitness level. The control unit may also allow a user to enter other variables such as whether the patient has bandages or clothing between the therapy wrap and the skin. Based on the user's answers to the prompts, the control unit selects a treatment protocol.

In various embodiments, the control unit displays a plurality of possible treatment parameters to the user. The user then selects one of the possible treatments. In various embodiments, the control unit may automatically select a treatment parameter from among the filter results based on the software programming.

At block 116, the user presses a “start” control and the control unit generates control signals to administer the treatment based on the selected treatment protocol. The start signal may also be generated by on time, an event, and other factors. For example, the system may start automatically at a preset time.

In the exemplary system, the control unit transmits control signals to pneumatic source 12, cooling source 15, power source 17, and pump 30 to deliver the desired treatment to the patient. In various embodiments, the control unit incorporates input data as shown in FIG. 4 and described above to adjust the treatment protocol for the conditions, such as the patient's characteristics and response.

Administration of treatment using the control unit at block 116 generally involves the following actions. Control unit 10 delivers pressurized gas to gas pressure bladder 28. The pressure in the gas pressure bladder exerts a compressive force on the body. In the exemplary system, once the gas pressure bladder is filled, the system generally maintains the pressure in the bladder by applying a backpressure.

Next the control unit activates pump 30 to circulate cooled fluid to fluid bladder 25. Circulation of the cooled fluid causes heat transfer with the body. In various embodiments, the thermal therapy device is operated under sufficient conditions to extract heat from a portion of an animate body. In various embodiments, the control unit delivers only compression therapy without heat exchange. The control unit may operate as described herein except that the heat transfer fluid is not circulated to the heat transfer device. Instead, the exemplary fluid bladder remains empty while the gas pressure bladder is filled. In this case, the therapy wrap may act like an air cast or splint.

In various embodiments, the control unit primes and calibrates system 5 before administering treatment. The optional priming and calibration operations may include a self-test to ensure that all the components are connected correctly. For example, the system can test for fluid or gas leaks. In various embodiments, the system includes a mechanical slide or electrical contacts that indicate to the user and/or control unit that all connections have been properly made or not. The system can also run a self-check to confirm the components are in working order. Once the system is applied to the body and ready, the system is primed for operation. The exemplary priming includes charging the cooling source, power source, and/or the pump. The system also optionally runs some heat transfer fluid and gas through the heat transfer device to clear out the internal volumes.

In various embodiments the thermal therapy device is operated under sufficient conditions to cool the core body temperature. In various embodiments the thermal therapy device is operated under sufficient conditions to lower the body temperature below 95 degrees Fahrenheit (F), below 90 degrees F., below 80 degrees F., below 70 degrees F., below 60 degrees F., or below 50 degrees F. In various embodiments, the thermal therapy device is operated under sufficient conditions to lower the body temperature to between about 90 degrees F. and about 94 degrees F. In various embodiments, the thermal therapy device is operated under sufficient conditions to limit substantial increases in the core body temperature and/or mild hyperthermia. In various embodiments, the thermal therapy device is operated under sufficient conditions to lower the body temperature in uniform increments. In various embodiments, the thermal therapy device is operated under sufficient conditions to lower the body temperature at a linear rate. In various embodiments, the body temperature is decreased by a few degrees each minute. In various embodiments, the body temperature is decreased by at least 5 degrees F. per minute, preferably between about 5 and about 20 degrees F. per minute, more preferably between about 5 and about 10 degrees F. per minute. In various embodiments, the body temperature is decreased by between about 2 and about 10 degrees F. per hour, preferably about 5 degrees F. per hour. In various embodiments, the thermal therapy device is operated under sufficient conditions to lower the body temperature at a first rate for a first period of time and then a faster second rate thereafter. In various embodiments, the thermal therapy device is operated under sufficient conditions to lower the body temperature at a first rate for a first period of time and then a slower second rate thereafter.

In various embodiments, the thermal therapy device is cycled through different treatment conditions. For example, the device may be treated at one temperature for a first period of time and then treated at a lower temperature for a second period of time. In various embodiments, the body is cooled gradually, maintained at a predetermined temperature, and then restored to normal temperature gradually. In various embodiments, the thermal therapy device is configured to induce hypothermia. The device may apply different levels of cooling in different regions or to different body parts. For example, the device may apply greater cooling to the chest area than the wrist area to lower the body's thermoregulation defenses. The device may gradually even out the difference in temperatures as the body approaches the desired internal body temperature.

The exemplary control unit operates under an endpoint is reached. In the case of body cooling therapy, the control operates the system until cooling of the body core has been achieved. Achievement of core cooling can be determined when the body temperature has reached a predetermined level. Core cooling can also be determined using known information in combination with the system operating conditions. For example, the user can input the patient's characteristics, and based on this information the system can determine that sufficient core cooling has been achieved using information related to the observed system performance. The patient characteristics may include height, weight, fitness level, condition or injury, and more. The information related to the system performance may include elapsed time, flow rate, temperature drop of the cooling source, and more.

In various embodiments, the therapy wrap runs until the user manually turns it off (e.g. using the control panel or disconnecting the wrap) or the cooling source has warmed above a threshold level where it cannot provide further cooling. In various embodiments, when the endpoint of the thermal therapy has been reached, the control unit stops the flow of gas and/or application of backpressure to the gas pressure bladder and circulation of fluid to the fluid bladder.

It has been found that in some applications the body can go into shock if the body temperature is dropped too quickly or returned to normal too quickly. In some cases, restoring the body to normal temperature can cause a rebound effect where blood rushes back to extremities and other areas. This can lead to the release of harmful toxins. In various embodiments, control unit is configured to decrease the body temperature gradually over time and maintain the body temperature at a reduced target temperature for a period of time. In various embodiments, the body temperature is gradually restored to its normal temperature. The core body temperature versus time thus appears as a U-shape with a wide bottom.

At block 118, if therapy wrap 20 was not recognized initially by control unit 10 at block 112, the system outputs an “unrecognized” signal. The system optionally displays an error message to the user. The error may be logged into the control unit memory.

At block 120, the control unit requests whether the user wishes to proceed with automatic treatment selection or manual selection. In exemplary system 5, it should be noted that the user may override control unit 10 at any time for manual control. The exemplary control unit also includes an automatic, instant shut-off as a safety feature.

If the user selects “automatic”, at block 124, the control unit makes a second attempt to select a treatment protocol. The control unit may compare the unique identifier and input information to the predefined treatment protocols. For example, the control unit may perform a similar process to the one described above except that it selects the closest “best” treatment protocol to the input set. For example, the control unit may select a treatment protocol associated with a treatment set for which most but not all of the input values match the actually received input values.

If the user selects “manual”, at block 126 the control unit generates control signals to administer treatment based on the user defined input parameters from user interface 50.

Throughout the operation of system 5, the exemplary system acquires data at block 128. The data acquisition is performed as described above. In addition to the information acquisition previously described, all the above processes and results may be logged into the control unit memory. Sensors 65 and/or signal source 66 may also be used to monitor the patient's vital signs during administration of treatment.

The exemplary memory may thus include other information, for example, the therapy wrap unique identifier, how the treatment protocol was determined, the treatment protocol, the actual treatment, deviation between the protocol and observed treatment, time stamps, and more. The system may be configured to store information related to the amount of heat (Watts) exchanged with the body. The amount of heat exchanged may be an estimate derived based on the flow rate, temperature delta in the wrap, and treatment time. The memory may also be used to store notes from the user or clinician, such as medical notes related to the treatment.

One of skill in the art will appreciate that a number of other features and modifications are within the scope of the invention. For example, administration of treatment using the control unit at blocks 116, 124, and/or 126 may comprise booting up the system from a previously used treatment program. The user can stop before a treatment protocol is complete and the control unit can start from the point where treatment stopped when the system is turned back on. In various embodiments, the control unit recognizes the therapy wrap as noted above and remembers that the treatment was previously stopped prematurely. The control unit can then automatically start from the prior stop point.

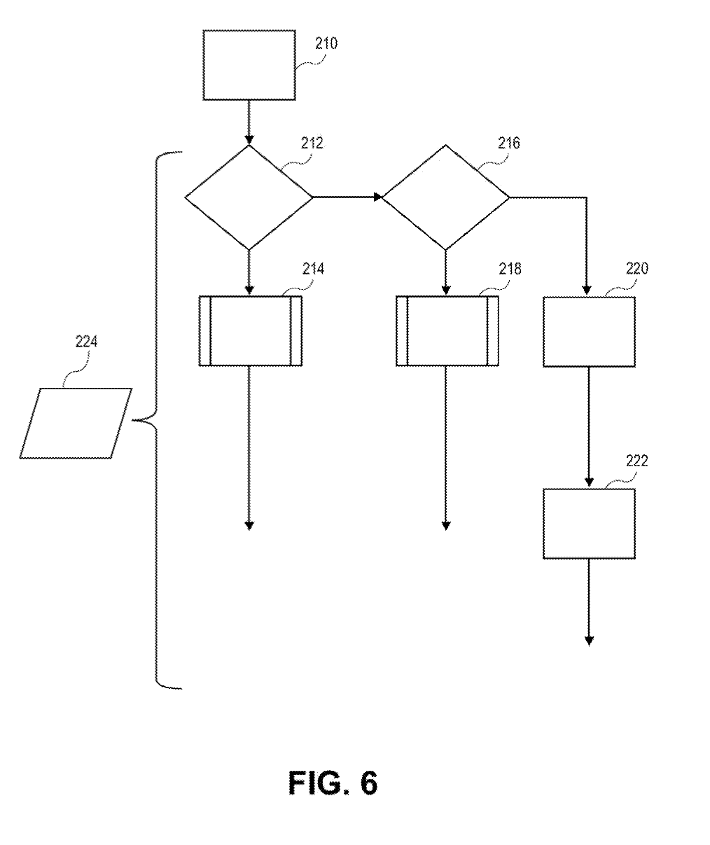

FIG. 6 illustrates a method of controlling thermal therapy similar to FIG. 5 above except the control unit operates based on waiting for a desired control signal. The method of FIG. 6 differs in that the control unit generally waits for a signal to perform a routine instead of selecting a routine based on the existing conditions. In other words, the method of FIG. 6 is a hybrid customized treatment control.

At block 210, control unit 10 requests a control signal.

At block 212, the control unit evaluates the control signal to determine if the control signal is a desired control signal. Suitable control signals may be a start operation from the user interface or a desired event. For example, therapy wrap 20 may include sensors on the attachment straps such that when the wrap is fastened to the user, a desired control signal is transmitted to the control unit. In the above example, the control unit would not receive a desired control signal before the therapy wrap is applied to the body. The control signal would become the desired control signal, however, once the straps are connected.

If the control unit received a desired control signal at block 212, the control unit administers a treatment based on a preset routine at block 214. Unlike the method illustrated in FIG. 5, in the exemplary method of FIG. 6 the control unit includes a defined, small set of preset treatment protocols. The treatment at block 214 is not performed until the desired control signal at block 212 is received.

If the desired control signal is not received at block 212, the control unit determines whether the control signal is another desired control signal at block 216. The comparison at block 216 is similar to the process at block 212 except the desired control signal is different.

If the control unit received a desired control signal at block 216, the control unit administers a treatment based on a preset routine at block 218. In the exemplary embodiment, the treatment routine at block 218 is different than the treatment routine at block 214.

If the received control signal does not match the desired control signal at block 216 either, the system prompts the user for control parameters at block 220. The system is then manually controlled in conventional fashion at block 222.

At block 224, the system acquires data during operation similar to the method described above.

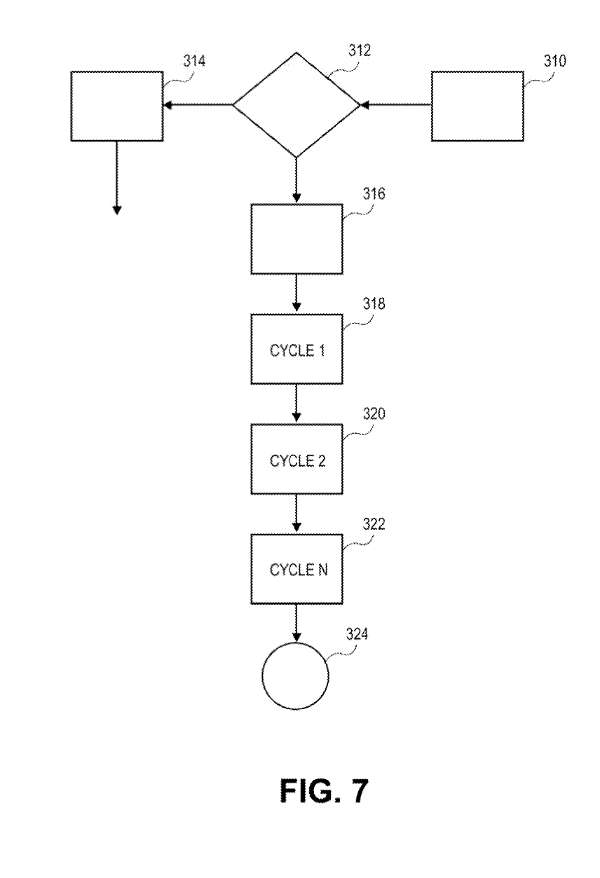

FIG. 7 illustrates a modified method of administering a thermal therapy treatment similar in various respects to the methods of FIGS. 5 and 6.

At block 310, the control unit receives a start signal.

At block 312, the control evaluates the received signal. The process at block 310 may include determining if the received signal is a desired control signal as described with reference to FIG. 6. The method illustrated in FIG. 7 may relate to a subcomponent of the control unit such as a subroutine. In this case, the subroutine is only performed when a desired control signal is received and remains inactive until the desired control signal is received. For example, the control unit may select a treatment protocol as described with reference to FIG. 5. The subroutine is then performed if the selected treatment protocol is a predetermined or desired treatment protocol.

At block 314, the system reverts to manual mode if the signal is not the desired signal.

At block 316, the control unit designates a selected treatment protocol.

At blocks 318, 320, and 322, the control unit turns on the pump to circulate fluid to the heat transfer device and activates the compression device in a series of steps. In various embodiments, the system is preprogrammed with compression treatment programs. For example, the system may be loaded with a program to deliver compression therapy in the form of alternating intervals of compression on and compression off for a specific injury.

At block 318, the control unit administers treatment under a first set of conditions. This step is referred to as a first cycle. Next, at block 320, the control unit administers treatment under a second set of conditions which are different than the second. This step is referred to as the second cycle. Next, at block 322, the control unit administers treatment under a first set of conditions which are different than the second. This step is referred to as the third cycle. Although described in terms of three cycles, one will appreciate that the method may be modified to have two cycles, four cycles, or more.

In various embodiments, the control unit changes the compression force in the first, second, and third conditions. In various embodiments, the control unit changes the heat transfer rate by modifying the flow rate and/or heat transfer fluid temperature at the therapy wrap inlet.

In various embodiments, the method is directed to treating a wound, either from injury or surgery. The first therapy conditions comprise a low compressive force and a treatment time of a few days. Once the wound has healed sufficiently to withstand compression, the compressive force is increased in the second cycle. Thereafter, the average temperature in the therapy wrap is allowed to fluctuate in the third cycle.

At block 324, the control unit stops treatment at an endpoint. In an exemplary embodiment, the system is shut down when a treatment times out.

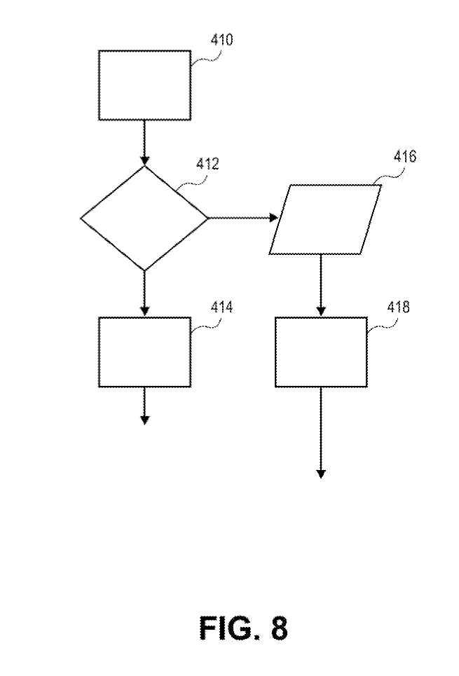

FIG. 8 is a flowchart illustrating an exemplary embodiment of a start-up routine of control unit 10 and system 5.

At block 410, the control unit starts in a manner similar to that described above.

At block 412, however, the control unit checks for whether an error signal was received during a previous treatment routine.

At block 414, if an error signal was generated during the previous routine, the control unit starts up in safe mode. In various embodiments, safe mode comprises operation of heat transfer device 22 without any, or significantly reduced, compression. In various embodiments, safe mode comprises operation for a reduced period of time.

At block 416, if no error signal was generated during the previous routine, the control unit starts up in normal mode. In various embodiments, the control unit performs the above operation and enters safe mode or normal mode automatically.

One of skill in the art will appreciate that a number of other features and modifications are within the scope of the invention.

Variations and modifications of any of the devices and methods disclosed herein will be readily apparent to persons skilled in the art. As such, it should be understood that the foregoing detailed description and the accompanying illustrations, are made for purposes of clarity and understanding, and are not intended to limit the scope of the invention, which is defined by the claims appended hereto. Any feature described in any one embodiment described herein can be combined with any other feature of any of the other embodiment whether preferred or not.

For convenience in explanation and accurate definition in the appended claims, the terms “up” or “upper”, “down” or “lower,” “inside” and “outside” are used to describe features of the present invention with reference to the positions of such features as displayed in the figures.

In many respects the modifications of the various figures resemble those of preceding modifications and the same reference numerals followed by apostrophes or subscripts “a”, “b”, “c”, and “d” designate corresponding parts.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A system for providing thermal treatment to an animate body requiring treatment, the system comprising:
a therapy wrap including at least one heat transfer device adapted to circulate a heat transfer fluid to exchange heat with the animate body; and
a control unit including:
an input device for receiving an input signal;
a processor for determining a treatment protocol based on the received input signal;
a controller for administering treatment to the animate body using the therapy wrap based on the determined treatment protocol.

2. The system of claim 1, wherein the processor determining includes comparing the received input signal to a desired input signal, and wherein the processor selects a treatment protocol if the received input signal is the desired input signal and selects another treatment protocol if the received input signal is not the desired input signal.

3. The system of claim 1, further comprising a heat transfer fluid source for supplying heat transfer fluid to the therapy wrap, wherein the controller regulates the rate of heat transfer by the heat transfer device based on the determined treatment protocol.

4. The system of claim 1, wherein the heat transfer device further comprises a fluid bladder including an inlet, an outlet, and at least one fluidic channel connecting the inlet to the outlet.

5. The system of claim 1, wherein the heat transfer device further comprises an expandable gas pressure bladder on a side of the fluid bladder opposite the animate body for exerting a compressive force on the bladder.

6. The system of claim 1, wherein the therapy wrap is a sleeve including a pouch for receiving the fluid bladder.

7. The system of claim 1, wherein the therapy wrap comprises a plurality of heat transfer devices, each of the plurality of heat transfer devices comprising a fluid bladder for circulating a heat transfer medium, the therapy wrap adapted to position the heat transfer devices adjacent the animate body at different locations.

8. A computer program product for use with a control unit having a computer processor and adapted to provide thermal therapy to an animate body using a therapy wrap, the computer program product comprising a computer readable storage medium and a computer program mechanism embedded therein, the computer program mechanism comprising:
an analyzer for recognizing a unique identifier associated with a therapy wrap.

9. The computer program product of claim 8, further comprising logic for selecting treatment settings based on the recognized unique identifier.

10. The computer program product of claim 8, further comprising:
logic for regulating thermal therapy of an animate body using the therapy wrap based on the treatment settings.

11. The computer program product of claim 10, further comprising:
logic for regulating pressure therapy of the animate body using the therapy wrap based on the treatment settings.

12. A method for treating an animate body in need of treatment, the method comprising:
applying a first therapy wrap to a portion of an animate body, the first therapy wrap comprising a heat transfer device adapted to circulate a heat transfer fluid to transfer heat with the animate body;
connecting the first therapy wrap to a control unit for regulating circulation of the heat transfer fluid in the heat transfer device;
treating the animate body using the first therapy wrap under a first set of treatment settings;
applying a second therapy wrap to a portion of an animate body, the second therapy wrap comprising a therapy device adapted to provide treatment to the animate body; and
treating the animate body using the second therapy wrap under a second set of treatment settings.

13. The method of claim 12, further comprising, before the applying a second therapy wrap, disconnecting the first therapy wrap from the control unit.

14. The method of claim 12, wherein the treating under the first set of treatment settings and the second set of treatment settings are the same.

15. The method of claim 12, wherein the under the first set of treatment settings and the second set of treatment settings are different.

16. The method of claim 12, wherein the first therapy device comprises a fluid bladder for circulating a heat transfer medium adapted to transfer heat with the animate body.

17. The system of claim 7, wherein the plurality of heat transfer devices includes a first heat transfer device adapted to cover the torso of the animate body and a second heat transfer device adapted to cover an extremity of the animate body, wherein the first heat transfer device is configured to cool the torso of the animate body and modulate the core temperature of the animate body and the second heat transfer device is configured to warm the extremity.

18. The system of claim 1, wherein the controller is configured to disable the system if authorization to use the system is not received by the controller.

19. The system of claim 18, wherein authorization to use the system includes payment information or authorization of payment information.

20. The system of claim 19, wherein the controller is configured to allow the system to be used for a predetermined time period.

21. The system of claim 18, wherein the controller is configured to allow the system or wrap to be used for a predetermined number of cycles.

22. The system of claim 18, wherein the controller is configured to permanently disable the system or wrap.

23. The system of claim 21, wherein the predetermined number of cycles is specified in a patient specific code that is entered before use.
